(19) United States
(12) Patent Application Publication
(10) Pub. No.: US 2015/0297652 A1
(43) Pub. Date: Oct. 22, 2015

(54) COMPOSITIONS AND METHODS FOR THEIR DERMATOLOGICAL USE

(71) Applicant: VITA NATURALE, LLC, St. Louis, MO (US)

(72) Inventor: Brundha Balaraman, St. Louis, MO (US)

(21) Appl. No.: 14/646,049

(22) PCT Filed: Nov. 19, 2013
(86) PCT No.: PCT/US13/70779

§ 371 Date: May 20, 2015

Related U.S. Application Data
(60) Provisional application No. 61/728,386, filed on Nov. 20, 2012.

(51) Int. Cl. A61K 36/074; A61K 36/489; A61K 36/185; A61K 36/58; A61K 36/79; A61K 36/74; A61K 36/539; A61K 36/53; A61K 36/882; A61K 36/282; A61K 36/48; A61K 36/23

(52) U.S. Cl. CPC A61K 36/074; A61K 36/48; A61K 36/489; A61K 36/185; A61K 36/58; A61K 36/23; A61K 36/74; A61K 36/539; A61K 36/53; A61K 36/882; A61K 36/282; A61K 36/79

(57) ABSTRACT

Compositions containing Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorns and/or Artemisia and/or Rheum and/or Schisandra and/or Ophiopogon japonicas and their use in preventing, treating, alleviating symptoms and/or mitigating skin disorders and/or disease are provided.

This patent application claims the benefit of priority from U.S. Provisional Application Ser. No. 61/728,386, filed Nov. 20, 2012, the teachings of which are herein incorporatred by reference in their entirety.

### FIELD OF THE INVENTION

The present invention relates to compositions containing Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicus. The present invention also relates to use of these compositions in preventing, treating, alleviating symptoms and/or mitigating skin disorders and/or disease.

### BACKGROUND OF THE INVENTION

Albizia lebbeck is a species of Albizia, fast-growing subtropical and tropical trees and shrubs, native to Indomalaya, New Guinea and Northern Australia. Lebbeck is an astringent and has been used by some cultures in the treatment or alleviation of boils, cough, eye disordors, flu symptoms, gingivitis, lung problems and pectoral problems. It has been suggested for use as an oral tonic in the treatment of abdominal tumors. (See, for example, Duke, Jame A., Dr. Duke's Phytochemical and Ethnobotanical Databases—Albizia lebbeck 2008). The bark is used medicinally to treat inflammation (Lowry et al. (1994): 2.5 Albizia lebbeck—a Promising Forage Tree for Semiarid Regions. In: Gutteridge, Ross C. and Shelton H. Mac (eds.):Forage Tree Legumes in Tropical Agriculture. CAB Intemational).

Sophora flavescens is a species of plant in the genus Sophora. About fifteen species in this genus have been used in traditional Chinese medicines. Common suggested uses include treatment of viral hepatitis, enteritis, cancer, viral myocarditis, gastrointestinal hemorrhage and skin diseases such as vaginitis, psoriasis and eczema.

Hibiscus rosa-sinensis, also known as rose mallow, Chinese hibiscus, China rose and shoe flower, is a species of flowering plant in the family Malvaceae. Hibiscus rosa-sinensis has also been used traditionally in Chinese medicines. An extract from the flowers of Hibiscus rosa-sinensis has been shown to function as an anti-solar agent by absorbing ultraviolet radiation (Nevade et al. 2011. Linn. Research Journal of Pharmacy and Technology 4(3): 472-473).

Phyllanthus emblica (syn. Emblica officinalis), is a deciduous tree of the family Phyllanthaceae. Preliminary research has shown in vitro antiviral and antimicrobial properties of its berries (Saeed, S. and Tariq, P. Pak J Pharm Sci 2007 20(1): 32-5). There is also preliminary evidence in vitro that its berry extracts induce apoptosis and modify gene expression in osteoclasts involved in rheumatoid arthritis and osteoporosis (Penolazzi et al. BMC Complementary and Alternative Medicine 2008 8: 59) and may prove to have activity against some cancers (Ngamkitidechakul et al. Phytotherapy Research 2010 24 (9): 1405-1413). A recent study in rats showed E. officinalis to reduce severity of acute pancreatitis and to promote spontaneous repair and regeneration of the pancreas occurring after an acute attack (Sidhu et al. Journal of Medicinal Food 2011 14 (1-2): 147-155). Preparations of leaves, bark and/or fruit have also shown potential efficacy against inflammation, cancer, age-related renal disease and diabetes in disease models (Ganju et al. Biomed Pharmacother 2003 57 (7): 296-300; Yokozawa et al. J Agric Food Chem. 2007 55(19): 7744-52; Rao et al. J Med Food 2005 8 (3): 362-8; and Qureshi et al. Pakistan Journal of Nutrition 2009 8 (2): 125-128) and a pilot study in humans showed a reduction of blood cholesterol levels in both normal and hypercholesterolemic men upon treatment with a berry extract (Jacob et al. Eur J Clin Nutr 1988 42 (11): 939-44).

Azadirachta indica, also referred to as NEEM, has been disclosed to have anti-bacterial, anti-parasitic, anti-fungal, anti-protozoal and anti-viral properties. NEEM, when applied in the form of a powder or oil has been disclosed to have exceptional results on external cuts or wounds and to to be very effective in relieving skin ailments such as eczema, acne, skin allergy, rashes, itch, ringworms, etc. NEEM water is also described as being very effective when used to treat burn injuries. In addition, NEEM oil is suggested to prevent hair from graying and to be effective in treating dandruff, lice and hair loss. NEEM oil has also been suggested for use in massaging muscle aches and joints to releive pain from conditions such as rheumatoid arthritis, gout, osteoarthritis and lower back pain. Ingesting NEEM is suggested to be beneficial in restoring taste, curing constipation and relieving indigestion. It also eliminates the problem of acidity. See soultemplenet with the extension .org/profiles/blogs/health-benefits-of-azadirachta of the world wide web.

Centella is used as a leafy green in various cuisines and is considered quite nutritious. However, while Centella asiatica has been promoted for its health benefits, available scientific evidence has not supported claims of its effectiveness for treating cancer or any other diseases in humans (“Gotu Kola” American Cancer Society. 28 Nov. 2011).

Ganoderma is a genus of polypore mushrooms which grow on wood, and include about 80 species. Collectively, the Ganoderma species are being investigated for a variety of potential therapeutic benefits including anticancer effects, immunoregulatory effects, antioxidant activities, liver-protecting effects, hypoglycemic effects, antibacterial effects, antiviral effects, antifungal effects and reducing blood cholesterol (Yuen, J W and Gohel, M. D. Nutr Cancer 2005 53 (1): 11-7; Xu et al. American Journal of Chinese Medicine 2011 39 (1): 15-27; Sliva D. Mini Reviews in Medicinal Chemistry 2004 4 (8): 873-9; and Sanodiya et al. Current Pharmaceutical Biotechnology 2009 10 (8): 717-42).

Rubia cordifolia, also known as Common Madder or Indian Madder, is a species of flowering plant in the coffee family, Rubiaceae. It has been cultivated for a red pigment derived from roots and has been described to have anti-inflammatory properties and induce urolithiasis in cellular and animal models (Joshan et al. Biomedical and Pharmacology Journal 2010 3:1 123-128; and Divakar et al. Food and Chemical Toxicology 2010 48:4 1013-1018).

Scutellaria baicalensis and Astragalus propinquus are species of flowering plants in the Lamiaceae family and family Fabaceae, respectively. Both are included in the 50 fundamental herbs used in traditional Chinese medicine. A. propinquus has been asserted to be a tonic that can improve the functioning of the lungs, adrenal glands and the gastrointestinal tract, increase metabolism and sweating, promote healing, and reduce fatigue (Balch, P. 2006 Prescription for Nutritional Healing (4th ed.) Avery Penguin Putnam).

Ocimum tenuiflorum, also known as Ocimum sanctum, Holy basil, or tulasi, is an aromatic plant in the family Lamiaceae. In vitro and animal studies have indicated some potential pharmacological properties of Ocimum tenuiflorum or its extracts as painkillers, antihyperlipidemics, cardioprotectants, anti-cancer agents and mitigants of the effects of radiation exposure, and antibacterial agents (Prakash, P. and Gupta, N. Indian Journal of Physiology and Pharmacology 2005 49 (2): 125-131; Suanarunsawat et al.

Journal of Basic and Clinical Physiology and Pharmacology 2010 21 (4): 387-400; Baliga et al. Nutrition and cancer 2013 65 Suppl 1: 26-35; and Golshahi et al. Clinical Biochemistry. Conference: 12th Iranian Congress of Biochemistry, ICB and 4th International Congress of Biochemistry and Molecular Biology 2011 44 (13 SUPPL. 1): S352).

Acorus is a genus of monocot flowering plants and Artemisia is a genus of plants including the sagebrush and wormwood.

Rheumis a genus of about 60 perennial plants in the family Polygonaceae. Many rheum species have food and medicinal uses dating back as far as 2000 years ago.

Oral administration of schisandra has been investigated for a number of conditions including hepatitis; improving mental and physical performance; increasing resistance to disease and stress; preventing aging; normalizing blood sugar and blood pressure; stimulating the immune system and speeding recovery after surgery; treating high cholesterol, coughs, asthma, sleep problems, nerve pain, premenstrual syndrome (PMS), chronic diarrhea, dysentery, night sweats, spontaneous sweating, involuntary discharge of semen, thirst, erectile dysfunction (ED), physical exhaustion, excessive urination, depression, irritability, and memory loss; improving vision; protecting against radiation, preventing motion sickness; preventing infection; boosting energy at the cellular level; counteracting the effects of sugar; and improving the health of the adrenal glands.

Ophiopogon japonicus, also known as Mondo grass, Fountain plant and monkey grass, is also used in traditional Chinese medicine. According to the Chinese Herbal Medicine Materia Medica, the herb is sweet, slightly bitter and slightly cold, enters the heart, lung and stomach channels and nourishes the yin of the stomach, spleen, heart and lungs and clears heat and quiets irritability.

### SUMMARY OF THE INVENTION

An aspect of the present invention relates to a composition comprising Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicus.

Another aspect of the present invention relates to a formulation for topical intralesional or oral administration comprising a composition comprising Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicus formulated in, for example, ointments, creams, lotions, solutions, pastes (e.g. facial mask), gels, emulsions, sprays, aerosols, oils, patches, toothpastes, mouthwashes, deodorants, soaps, body and/or face washes, sponges, foams, semi-solids, cosmetics (e.g. solid, semisolid, liquid, or powder foundation, eye shadow, lip balm), application sticks, sunscreens, depots, suppositories, sustained-release formulations (a unique variation of this concept presented herein involves tea bag-like carriers made of plastic, silk, nylon, Soilon or paper), troches, tablets, pills, capsules, syrups, elixirs, suspensions, wafers, foods (e.g. chewing gum, mints, etc.), beverages or other formulations which accomplish direct contact between the composition of the present invention and a cutaneous or mucosal surface or lesion.

Another aspect of the present invention relates to a method of preventing, mitigating, and/or treating skin disorders and/or diseases by administering to a subject having or suspected of having a skin disorder and/or disease a composition comprising Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicus.

### DETAILED DESCRIPTION OF THE INVENTION

The present invention relates to compositions comprising Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicus.

In one embodiment, the composition of the present invention comprises Albizia lebbeck and one or more agents selected from the group consisting of Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Ocimum tenuiflorum (syn. Ocimum sanctum) and/or Acorus and/or Artemisia and/or Rheum and/or Schisandra and/or Ophiopogon japonicus.

In another embodiment, the composition of the present invention comprises Sophora flavescens and one or more agents selected from the group consisting of Albizia lebbeck and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Ocimum tenuiflorum (syn. Ocimum sanctum) and/or Acorus and/or Artemisia and/or Rheum and/or Schisandra and/or Ophiopogon japonicus.

In another embodiment, the composition comprises Hibiscus rosa sinensis and one or more agents selected from the group consisting of Albizia lebbeck and/or Sophora flavescens and/or Phyllanthus emblica (syn. Emblica officinalis) and/or Azadirachta indica and/or Centella asiatica and/or Ganoderma and/or Rubia cordifolia and/or Scutellaria baicalensis and/or Ocimum tenuiflorum (syn. Ocimum sanctum) and/or Acorus and/or Artemisia and/or Rheum and/or Schisandra and/or Ophiopogon japonicus.

In another embodiment, the composition comprises Phyllanthus emblica (syn. Emblica officinalis) and one or more agents selected from the group consisting of Albizia lebbeck and/or Sophora flavescens and/or Hibiscus rosa sinensis and/or Azadirachta indica and/or Centella asiatica and/or Ganoderma and/or Rubia cordifolia and/or Scutellaria baicalensis and/or Ocimum tenuiflorum (syn. Ocimum sanctum) and/or Acorus and/or Artemisia and/or Rheum and/or Schisandra and/or Ophiopogon japonicus.

In yet another embodiment, the composition of the present invention comprises Azadirachta indica and one or more agents selected from the group consisting of Albizia lebbeck and/or Sophora flavescens and/or Hibiscus rosa sinensis and/or Phyllanthus emblica (syn. Emblica officinalis) and/or Centella asiatica and/or Ganoderma and/or Rubia cordifolia and/or Scutellaria baicalensis and/or Ocimum tenuiflorum (syn. Ocimum sanctum) and/or Acorus and/or Artemisia and/or Rheum and/or Schisandra and/or Ophiopogon japonicus.

In yet another embodiment, the composition of the present invention comprises Centella asiatica and one or more agents selected from the group consisting of Albizia lebbeck and/or Sophora flavescens and/or Hibicus rosa sinensis and/or Phyllanthus emblica (syn. Emblica officinalis) and/or Azadirachta indica and/or Ganoderma and/or Rubia cordifolia and/or Scutellaria baicalensis and/or Ocimum tenuiflorum (syn. Ocimum sanctum) and/or Acorus and/or Artemisia and/or Rheum and/or Schisandra and/or Ophiopogon japonicus.

In yet another embodiment, the composition of the present invention comprises Ganoderma and one or more agents selected from the group consisting of Albizia lebbeck and/or Sophora flavescens and/or Hibiscus rosa sinensis and/or Phyllanthus emblica (syn. Emblica officinalis) and/or Azadirachta indica and/or Centella asiatica and/or Rubia cordifolia and/or Scutellaria baicalensis and/or Ocimum tenuiflorum (syn. Ocimum sanctum) and/or Acorus and/or Artemisia and/or Rheum and/or Schisandra and/or Ophiopogon japonicus.

In yet another embodiment, the composition of the present invention comprises Rubia cordifolia and one or more agents selected from the group consisting of Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Scutellaria baicalensis and/or Ocimum tenuiflorum (syn. Ocimum sanctum) and/or Acorus and/or Artemisia and/or Rheum and/or Schisandra and/or Ophiopogon japonicus.

In yet another embodiment, the composition of the present invention comprises Scutellaria baicalensis and one or more agents selected from the group consisting of Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Ocimum tenuiflorum (syn. Ocimum sanctum) and/or Acorus and/or Artemisia and/or Rheum and/or Schisandra and/or Ophiopogon japonicus.

In yet another embodiment, the composition of the present invention comprises Astragalus membranaceus and one or more agents selected from the group consisting of Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Ocimum tenuiflorum (syn. Ocimum sanctum) and/or Acorus and/or Artemisia and/or Rheum and/or Schisandra and/or Ophiopogon japonicus.

In yet another embodiment, the composition of the present invention comprises Ocimum tenuiflorum (syn. Ocimum sanctum) and one or more agents selected from the group consisting of Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Acorus and/or Artemisia and/or Rheum and/or Schisandra and/or Ophiopogon japonicus.

In yet another embodiment, the composition of the present invention comprises Acorus and one or more agents selected from the group consisting of Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Ocimum tenuiflorum (syn. Ocimum sanctum) and/or Artemisia and/or Rheum and/or Schisandra and/or Ophiopogon japonicus.

In yet another embodiment, the composition of the present invention comprises Artemisia and one or more agents selected from the group consisting of Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Ocimum tenuiflorum (syn. Ocimum sanctum) and/or Acorus and/or Rheum and/or Schisandra and/or Ophiopogon japonicus.

In yet another embodiment, the composition of the present invention comprises Rheum and one or more agents selected from the group consisting of Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Ocimum tenuiflorum (syn. Ocimum sanctum) and/or Acorus and/or Artemisia and/or Schisandra and/or Ophiopogon japonicus.

In yet another embodiment, the composition of the present invention comprises Schisandra and one or more agents selected from the group consisting of Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Ocimum tenuiflorum (syn. Ocimum sanctum) and/or Acorus and/or Artemisia and/or Rheum and/or Ophiopogon japonicus.

In yet another embodiment, the composition of the present invention comprises Ophiopogon japonicus and one or more agents selected from the group consisting of Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Ocimum tenuiflorum (syn. Ocimum sanctum) and/or Acorus and/or Artemisia and/or Rheum and/or Schisandra.

In one embodiment, a composition of the present invention is formulated for topical administration to a cutaneous or mucosal surface.

In another embodiment, a composition of the present invention is formulated for intralesional administration to a cutaneous or mucosal surface.

In yet another embodiment, a composition of the present invention is formulated for oral administration to a mucousal surface.

Examples of topical, intralesional and oral formulations include, but are not limited to, ointments, creams, lotions, solutions, pastes (e.g. facial mask), gels, emulsions, sprays, aerosols, oils, patches, toothpastes, mouthwashes, deodorants, soaps, body and/or face washes, sponges, foams, semi-solids, cosmetics (e.g. solid, semisolid, liquid, or powder foundation, eye shadow, lip balm), application sticks, sunscreens, depots, suppositories, sustained-release formulations (a unique variation of this concept presented herein involves tea bag-like carriers made of plastic, silk, nylon, Soilon or paper), troches, tablets, pills, capsules, syrups, elixirs, suspensions, wafers, foods (e.g. chewing gum, mints, etc.), beverages or other formulations which accomplishes direct contact between the composition of the present invention and a cutaneous or mucosal surface or lesion.

The compositions of the present invention are useful in preventing, treating, alleviating symptoms and/or mitigating skin disorders and/or diseases. Examples of such skin disorders and/or diseases include, but are not limited to, neoplasms, infections, disorders of inflammation, proliferation, autoimmunity, hypersensitivity, pruritus, photodamage or photoaging, wound healing, angiogenesis, hair, nails, odor or hyperhidrosis, and pigmentation. In one embodiment, a composition of the present invention is applied topically to prevent, treat, alleviate symptoms and/or mitigate the skin disorder and/or disease. In another embodiment, a composition of the present invention is applied intralesionally to treat alleviate symptoms and/or mitigate the skin disorder and/or disease. In another embodiment, a composition of the present invention is administered orally to treat alleviate symptoms and/or mitigate the skin disorder and/or disease. In these embodiments, the topical, intralesional or oral formulation of the present invention may further comprise one or more additional pharmaceutical ingredients established for use in a skin disorder or disease.

### Hair Disorders

For formulations of the present invention for use in hair disorders, in addition to Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicus, the formulation of the present invention may further comprise, for example, one or more established hair growth effectors such as, but not limited to, minoxidil, eflornithiquinone, prostaglandin agonists or antagonists (e.g. brimatoprost and latanoprost), 5-alpha reductase inhibitors (e.g. finasteride, dutasteride) and other anti-androgenic agents (e.g. spironolactone, flutamide), anti-inflammatory agents (e.g. corticosteroids or calcineurin inhibitors), hormones (e.g. androgens, estrogens), apigenin as well as additional herbal ingredients such as Eclipta prostrata (syn. Eclipta alba), Carthamus tinctorius,

Schisandra nigra, Polygonum multiflorum (syn. Fallopia multiflora), Trigonella foenum-graecum, Sapindus mukorossi, Thuja occidentalis, Herpestis Monniera, Bacopa monnieri, Acacia concinna, and other relevant ingredients listed herein.

### Antioxidant/Anti-Aging

For formulations of the present invention for use as antioxidants in disorders of photodamage and photoaging, in addition to Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicus, the formulation of the present invention may further comprise, for example, one or more of established antioxidant or anti-aging therapies which include, but are not limited to retinol, retinoids and derivatives thereof (e.g. adapalene, tretinoin, isotretinoin, acitretin, tazarotene), hydroquinone, and active sunscreen ingredients (e.g. aminobenzoic acid, avobenzone, anthranilates, cinnamates, octinoxate, cinoxate, benzophenones, dioxybenzone, Oxybenzone, Homosalate, Octocrylene, Octyl methoxycinnamate, Octisalate, Mexoryl SX, PABA derivatives, benzoic acid, Padimate O, Phenylbenzimidazole sulfonic acid, Sulisobenzone, Titanium dioxide, Trolamine salicylate, Zinc oxide). Examples of antioxidants include, but are not limited to ascorbyl palmitate, ascorbic acid (vitamin C), tetrahexyldecyl ascorbate, vitamin A, vitamin E acetate, ferulic acid, alpha-lipoic acid, especially DL-alpha-lipoic acid, biotin, folic acid, coenzyme Q10, glutathione, tripeptides and peptides, lipochroman-6, magnesium ascorbyl phosphate, (−)-epigallocatechin-3-gallate, catechins, galangin, rutin, luteolin, morin, fisetin, silymarin, Coenzyme Q10, apigenin, gingkolides, hesperidin, citrin, caffeine and caffeine salts, niacinamide, nicotinamide, xanthines, anthocyanins (e.g. pelargonidin 3-glucoside, cyanidin 3-glucoside, delphinidin 3-glucoside, pelargonidin 3,5-diglucoside, cyanidin 3,5-diglucoside and delphinidin 3,5-diglucoside), ellagitannins, hydrolysable tannins (e.g. punicalin, pedunculagin, punicalagin, gallagic and ellagic acid esters of glucose), and derivatives thereof which exhibit antioxidant activity. Antioxidant enzymes are further contemplated for use in the compositions of the present invention, such as superoxide dismutase, catalase, glutathione peroxidase, methionine reductase, and equivalents thereof. These antioxidant enzymes can prevent the formation of free radicals or scavenge the formed free radicals to prevent cell damage. Flavonoids and/or flavonoid derivatives are also contemplated for inclusion in compositions of the present invention. Examples of flavonoids and/or flavonoid derivatives and/or isoflavones include, but are not limited to quercetin, quercetrin, myricetin, kaempferol, myrecetrin and genistein as these compounds also have some anti-inflammatory activity and/or can help stabilize cell membranes in combination with relatively low toxicity. Also, pharmaceutically acceptable salts of these flavonoids and/or flavonoid derivatives may be employed. The particular flavonoid and/or flavonoid derivative included in the composition can be determined by factors such as toxicity, bioavailability, solubility or dispersability, among others. Additional herbal ingredients which can be included in formulations of the present invention for use as antioxidants in disorders of photodamage and photoaging include, but are not limited to Glycyrrhiza glabra, Pterocarpus santalinus, Vitis vinifera, Punica granatum, Polygonum multiflorum (syn. Fallopia multiflora), Soy, Olea europaea, Cinnamomum cassia, Momordica charantia, Phoenix Dactylyfera, Poria cocos, Syzygium cumini, Trigonella foenum-graecum, Terminalia chebula, Cucurbita pepo, Moringa oleifera, Tinospora cordifolia, Cajanus cajan, Kinobeon A, Santalum album, Rubia cordifolia, Lonicera caerulea, Vaccinium myrtillus, Hibiscus sabdariffa, Iris Florentina Root, Arbutin, Pyrus, Oenothera biennis, Aframomum angustifolium, Plumbago zeylanica, Polypodium leucotomos, Hemidesmus indicus, Terminalia bellerica, Caesalpinia crista, Nardostachys jatamansi, Salix nigra, Rosmarinus officinalis (e.g. Rosemary Oleoresin), Camellia sinensis, Hamamelis virginiana, Mangifera indica, Ginkgo biloba, Cocos nucifera (e.g. Coconut endosperm), Piper nigrum (e.g. tetrahydropiperine), Terminalia arjuna, Terminalia muelleri, Thuja standishii, Crocus sativus, Sesamum indicum, Cardamom (genera Elettaria and Amomum), Ligusticum wallichii (Tetramethylpyrazine), Phellodendron amurense, Pueraria lobata, Eucommia ulmoides, Ocimum basilicum, Bacopa monniera, Aloysia triphylla, Prunella vulgaris, Arctium lappa, Angelica dahurica, Forsythia suspense, Salvia miltiorrhiza, Mesua ferrea, Malabathrum, Cucumis satius, Broussonetia papyrifera, Morus bombycis and Tanacetum parthenium. Additional active agents which can be included in formulations of the present invention for use as antioxidants or in anti-aging include, but are not limited to, lycopene, resveratrol, α-carotene, β-carotene, L-ergothioneine, kojic acid, N-acetylglucosamine, hyaluronic acid, alpha-hydroxy acids (e.g. glycolic acid, lactic acid, citric acid), beta-hydroxy acids (e.g salicylic acid), trichloroacetic acid, bisabolol (syn. levomenol), amber extract, colhibin, L-Carnitine, exfoliants (e.g. aluminum oxide crystals), kinetin, statins, peptides (e.g. argireline and copper peptides), resorcinol, solid carbon dioxide slush, phenol, Isolagen, bromelain and other relevant ingredients listed herein. In one embodiment, the composition is administered topically, intralesionally or orally before, after or simultaneously with photodynamic therapy, dermabrasion, ablative and non-ablative lasers (e.g. pulsed dye laser, Erb:YAG, CO2, Nd:YAG, Fraxel, Q-switched, Diode, Argon, Pulse excimer, Krypton, Copper, V-beam), intense pulsed light (syn. IPL), plasmakinetic rejuvenation, photo-pneumatic technology, electro-optical synergy, or electrosurgery.

### Antimicrobial

For formulations of the present invention for use as antimicrobial agents, in addition to Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicus, the formulation of the present invention may further comprise, for example, one or more of established antimicrobial therapies which include, but are not limited to antibiotics (e.g. clindamycin, metronidazole, mupirocin, polysporin, gentamycin), anti-fungal agents (e.g. ketoconazole, nystatin, econazole, terbinafine, griseofulvin, ciclopirox, miconazole, thymol, zeasorb, nafitine, amphotericin), antivirals (e.g. acyclovir, docosanol, penciclovir, valacyclovir, cidofovir, foscarnet, imiquimod, canthacur, squaric acid), scabicides (e.g. permethrin, ivermectin, lindane, malathion), pediculicides, anti-helmintic (e.g. benzimidazoles, diethylcarbamazine, ivermectin, pyrantel pamoate, levamisole), resiquimod, benzoyl peroxide, silvadene, pentavalent antimony (e.g. stibogluconate), salicyclic acid, sulfur, sulfacetamide, pyrithione zinc, selenium sulfide, vinegar, and other relevant ingredients listed herein. Additional herbal ingredients which can be included in formulations of the present invention for use as antimicrobial agents include, but are not limited to Hemidesmus indicus, Melaleuca alternifolia, Hydrastis canadensis, Mahonia aquifolium, Andrographis paniculata, Manuka honey (e.g. methylglyoxal), Agastache rugosa, Dichroa febrifuga, Tridax procumbens, Lobelia chinensis, Ocimum basilicum, Melia azedarach, Aloysia triphylla, Acacia concinna, Rhus javanica, Picea abies (syn. Norway spruce), and Santalum album.

### Anti-Neoplastic

For formulations of the present invention for use as anti-neoplastic agents, in addition to Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicus, the formulation of the present invention may further comprise, for example, one or more of established anti-neoplastic therapies which include, but are not limited to cytotoxic agents such as TAXOL, Cytochalasin B, Gramicidin D, Ethidium Bromide, Emetine, Mitomycin, Etoposide, Tenoposide, Vincristine, Vinblastine,

Colchicin, Doxorubicin, Daunorubicin, Mitoxantrone, Mithramycin, Actinomycin D, 1-Dehydrotestosterone, Glucocorticoids, Procaine, Tetracaine, Lidocaine, and Puromycin and analogs or homologs thereof. Therapeutic agents include, but are not limited to, antimetabolites (e.g., Methotrexate, 6-Mercaptopurine, 6-Thioguanine, Cytarabine, 5-Fluorouracil, Decarbazine), alkylating agents (e.g., Mechlorethamine, Thiotepa, Chlorambucil, Melphalan, Carmustine (BCNU), Lomustine (CCNU), Cyclophosphamide, Busulfan, Dibromomannitol, Streptozotocin, Mitomycin C, Cis-Dichlorodiamine Platinum (II) (DDP), Cisplatin), anthracyclines (e.g., Daunorubicin (formerly Daunomycin) and Doxorubicin), antibiotics (e.g., Dactinomycin (formerly Actinomycin), Bleomycin, Mithramycin, and Anthramycin (AMC)), anti-mitotic agents (e.g., Vincristine and Vinblastine) and new agents such as Selective Apoptotic Antineoplastic Drugs (SAANDs) such as APTOSYN® (Exisulind). Additional active agents which can be included in formulations of the present invention for use in the treatment of neoplastic conditions include, but are not limited to imiquimod, diclofenac (e.g. Solaraze), interferon, ipilimumab, BRAF-inhibitors (e.g. vemurafenib), MEK-inhibitors, PD-1 inhibitors, nitrogen mustard, bexarotene, timolol or propanolol, corticosteroids, ingenol mebutate, rapamycin, sirolimus, imatinib, oxymetazoline hydrochloride, aminolevulinic acid (syn. Levulan), methyl aminolevulinic acid (syn. METVIX), hedgehog pathway inhibitors (e.g. vismodegib, syn. Erivedge™), resiquimod, and other relevant ingredients listed herein. Additional herbal ingredients which can be included in formulations of the present invention for use as antimicrobial agents include, but are not limited to Punica granatum, Terminalia Arjuna, Tinospora cordifolia, Rubia cordifolia, Gelsemium sempervirens, Thuja standishii, Aegle marmelos, Scutellaria barbata, Phellodendron amurense, Momordica charantia, Angelica dahurica, and Perilla frutescens. As will be understood by the skilled artisan upon reading this disclosure, many of the agents listed above are more effective when administered systemically. Accordingly, an aspect of the present invention also relates to use of a composition of the present invention topically, intralesionally or orally in combination with systemic administration of an established antineoplastic treatment for the treatment of a neoplastic skin disorder to disease. Compositions of the present invention can be administered alone or in combination with agents established to relieve effects of cancer and/or side effects of cancer before, simultaneously with, or following, the administration of a composition of the present invention. Examples of agents which relieve side effects of cancer include, but are not limited to, Epoetin alfa to relieve symptoms of anemia; cell protecting agents such as amifostine; and Strontium-89 and Samarium-153 for the relief of cancer-induced bone pain; and oral antibiotics (e.g. minocycline), corticosteroids, keratolytics, and retinoids for the relief of drug induced acneiform and papulosquamous eruptions.

### Anti-Proliferative

For formulations of the present invention for use as anti-proliferative agents, in addition to Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicus, the formulation of the present invention may further comprise, for example, one or more of established anti-neoplastic therapies which include, but are not limited to retinol or retinoids and derivatives thereof (e.g., isotretinoin, acitretin, adapalene, tretinoin, tazorac), vitamin D analogs (e.g. calcitriol, calcipotriene), calcineurin inhibitors (e.g. tacrolimus, pimecrolimus), corticosteroids (e.g. clobetasol, betamethasone, triamcinolone, hydrocortisone, prednisone), keratolytics (e.g. urea, azelaic acid, salicylic acid, lactic acid, and other alpha- or beta-hydroxyacids), nicotinamide, gentian violet, vincristine or vinblastine, liquor carbonis detergens, nitrogen mustard, bexarotene, alpha- or beta-agonists or antagonists (e.g. timolol or propranolol), calcium channel blockers, 5-fluorouracil, imiquimod, sinecatechins (e.g. Veregen), canthacur, squaric acid, podofilox, podophyllin, benzoin, ingenol mebutate, rapamycin, bleomycin, intralesional candida antigen, cidofovir, imatinib, oxymetazoline hydrochloride, aminolevulinic acid (syn. Levulan), hedgehog pathway inhibitors (e.g. vismodegib, syn. Erivedge™), emollients (e.g. ceramides), coal tar, tar gel, tar shampoo, antibiotics (e.g., doxycycline, metronidazole, trimethoprim-sulfamethaxole, cephalexin), anthralin, anti-tumor-necrosis-factor inhibitors (e.g. adalimumab, etanercept, infliximab), methotrexate, diaminodiphenyl sulfone (syn. dapsone), hydroxychloroquine (syn. plaquenil), chloroquine, quinacrine, azathioprine (syn. Imuran), anakinra (syn. Kineret), omalizumab (syn. Xolair), cyclosporine (syn. Neoral), cyclophosphamide (syn. Cytoxan), mycophenolate mofetil (syn. Cellcept or Myfortic), thalidomide, rituximab, ustekinumab (syn. stelara), alefacept, leflunomide, methoxypsoralen (syn. oxsoralen), immunoglobulins, sulfasalazine, mesalamine, bromelain, brodalumab and other immunomodulators and immunosuppressants and other relevant ingredients listed herein. Additional herbal ingredients which can be included in formulations of the present invention for use as anti-proliferative agents include, but are not limited to Punica granatum, Terminalia arjuna, Tinospora cordifolia, Rubia cordifolia, and Malabathrum. In one embodiment, the composition of the present invention is administered before, after or simultaneously with ultraviolet B radiation, ultraviolet A radiation, photodynamic therapy, dermabrasion, ablative and non-ablative lasers (e.g. pulsed dye laser, Erb:YAG, CO2, Nd:YAG, Fraxel, Q-switched, Diode, Argon, Pulse excimer, Krypton, Copper), intense pulsed light (syn. IPL), electrosurgery, administration of a chemical peeling agent (e.g. trichloroacetic acid, glycolic acid, salicyclic acid, recorcinol, lactic acid, phenol, croton oil) or sclerosing agents (e.g. hypertonic saline, sodium tetradecyl sulfate, SOTRADECOL™, polidocanol, sodium morrhuate, ethanolamine oleate, glycerin, polyiodine iodine), plasmakinetic rejuvenation, photo-pneumatic technology, electro-optical synergy, electrosurgery, and/or administration of a vasoconstrictor (e.g. catecholamines, oxymetazoline, phenylephrine, caffeine, theophylline, epinephrine, ergot compounds, triptans), penicillamine, tetracycline, cytokine (e.g. IL-12), matrix metalloproteinase inhibitor (e.g. batimastat and marimastat), direct angiogenesis inhibitor (e.g. bevacizumab, vascular endothelial growth factor receptor antagonists, angiostatin, basic fibroblast growth factor), or indirect angiogenesis inhibitor (e.g. Ras-signaling inhibitors, farnesyltranferase inhibitors).

### Disorders of Pigmentation

For formulations of the present invention for use in disorders of pigmentation, in addition to Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicus, the formulation of the present invention may further comprise, for example, one or more of established pigmentation therapies which include, but are not limited to kojic acid, lignin peroxidase, belides, soy extracts, niacinamide, N-acetylglucosamine, aloesin, bleaching agents (e.g. hydroquinone, benoquin), benzoyl peroxide, retinol or retinoids and derivatives thereof (e.g., isotretinoin, acitretin, adapalene, tretinoin, tazorac), active sunscreen ingredients (e.g. aminobenzoic acid, avobenzone, anthranilates, cinnamates, octinoxate, cinoxate, benzophenones, dioxybenzone, Oxybenzone, Homosalate, Octocrylene, Octyl methoxycinnamate, Octisalate, Mexoryl SX, PABA derivatives, benzoic acid, Padimate O, Phenylbenzimidazole sulfonic acid, Sulisobenzone, Titanium dioxide, Trolamine salicylate, Zinc oxide), alpha-Arbutin (syn. 4-Hydroxyphenyl-α-D-glucopyranoside), calcineurin inhibitors (e.g. tacrolimus, pimecrolimus), corticosteroids (e.g. clobetasol, betamethasone, triamcinolone, hydrocortisone, prednisone), keratolytics (e.g. urea, azelaic acid, salicylic acid, lactic acid, and other alpha- or beta-hydroxyacids), nicotinamide, bromelain, caffeine and derivatives thereof. Additional herbal ingredients which can be included in formulations of the present invention for use in pigmentation disorders include, but are not limited to Glycyrrhiza glabra, Morus alba, Syzygium aromaticum, Punica granatum, Arbutin, Cinnamomum cassia, Citrus aurantium, Oenothera biennis, Trigonella foenum-graecum, Withania somnifera, Atractylodes, Platycodon graniflorus, Angelica dahurica, Cucumis Satius, Broussonetia kazinoki, Broussonetia papyrifera, Perilla frutescens, Cornus officinalis, Rhus javanica, Morus bombycis, Quercus dentate, Schizopepon bryoniaefolius, Pinus densiflora, and Capsella bursa-pastoris. In one embodiment, the composition is administered topically, intralesionally or orally before, after or simultaneously with ultraviolet B radiation, ultraviolet A radiation, photodynamic therapy, dermabrasion, ablative and non-ablative lasers (e.g. pulsed dye laser, Erb:YAG, CO2, Nd:YAG, Fraxel, Q-switched, Diode, Argon, Pulse excimer, Krypton, Copper), intense pulsed light (syn. IPL), electrosurgery, chemical peeling agents (e.g. trichloroacetic acid, glycolic acid, salicyclic acid, recorcinol, lactic acid, phenol, croton oil), and other relevant ingredients listed herein.

### Anti-Inflammatory

For formulations of the present invention for use as anti-inflammatory agents, in addition to Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicus, the formulation of the present invention may further comprise, for example, one or more of established anti-inflammatory therapies which include, but are not limited to pycnogenol, oatmeal, avenanthramides, bisabolol (syn. Levomenol), D- and L-Panthenol, azelaic acid, non-steroidal anti-inflammatory drugs (e.g. aspirin, ibuprofen, piroxicam), statins, chlorambucil, acetaminophen, allopurinol, hydroxyurea, penicillamine, cromolyn sodium, colchicine, diclofenac, retinol or retinoids and derivatives thereof (e.g., isotretinoin, acitretin, adapalene, tretinoin, tazorac), vitamin D analogs (e.g. calcitriol, calcipotriene), calcineurin inhibitors (e.g. tacrolimus, pimecrolimus), corticosteroids (e.g. clobetasol, betamethasone, triamcinolone, hydrocortisone, prednisone), keratolytics (e.g. urea, azelaic acid, salicylic acid, lactic acid, and other alpha- or beta-hydroxyacids), liquor carbonis detergens, nicotinamide, gentian violet, vincristine or vinblastine, nitrogen mustard, bexarotene, rapamycin, bleomycin, cidofovir, imatinib, oxymetazoline hydrochloride, hedgehog pathway inhibitors (e.g. vismodegib,syn. Erivedge™), emollients (e.g. ceramides), coal tar, tar gel, tar shampoo, rifampin, antibiotics (e.g., doxycycline, metronidazole, trimethoprim-sulfamethaxole, cephalexin), anthralin, anti-tumor-necrosis-factor inhibitors (e.g. adalimumab, etanercept, infliximab), methotrexate, diaminodiphenyl sulfone (syn. dapsone), hydroxychloroquine (syn. plaquenil), chloroquine, quinacrine, azathioprine (syn. Imuran), anakinra (syn. Kineret), omalizumab (syn. Xolair), cyclosporine (syn. Neoral), cyclophosphamide (syn. Cytoxan), mycophenolate mofetil (syn. Cellcept, Myfortic), thalidomide, rituximab, ustekinumab (syn. stelara), alefacept, leflunomide, methoxypsoralen (syn. oxsoralen), immunoglobulins, sulfasalazine, mesalamine, bromelain, brodalumab and other immunomodulators and immunosuppressants. Additional herbal ingredients which can be included in formulations of the present invention for use as anti-inflammatory agents include, but are not limited to, Terminalia chebula (e.g. chebulagic acid), Embelia ribes, Vitex negundo, Picrorhiza kurroa, Pterocarpus santalinus, Punica granatum, Terminalia arjuna, Tinospora cordifolia, Rubia cordifolia, Eclipta alba (syn. eclipta prostrata), Rosmarinus officinalis, Boswellia serrate, Citrus aurantium, Cinnamomum cassia, Trigonella foenum-graecum, Pyrus, Lonicera caerulea, Vaccinium myrtillus, Diosgenin, Cocos nucifera, Mahonia aquifolium, Andrographis paniculata, Aegle marmelos, Fritillaria thunbergii, Calendula, Crocus sativus, Coptis, Cardamom (Genera Elettaria and Amomum), Dichroa febrifuga, Leonurus japonicus, Ligusticum wallichii (e.g. tetramethylpyrazine), Lobelia chinensis, Phellodendron amurense, Rehmannia glutinosa, Eucommia ulmoides, Gleditsia sinensis, Trichosanthes, Melia azedarach, Astragalus membranaceus, Platycodon graniflorus, Arctium lappa, Forsythia suspense, Salvia miltiorrhiza, Leonurus sibiricus, Mesua ferrea, Malabathrum, Bacopa monnieri, Broussonetia kazinoki, Perilla frutescens, Morus bombycis, Camellia sinensis, Glycyrrhiza glabra and Tanacetum parthenium. In one embodiment, the composition is administered topically, intralesionally or orally before, after or simultaneously with ultraviolet B radiation, ultraviolet A radiation, photodynamic therapy, dermabrasion, ablative and non-ablative lasers (e.g. pulsed dye laser, Erb:YAG, CO2, Nd:YAG, Fraxel, Q-switched, Diode, Argon, Pulse excimer, Krypton, Copper, V-beam), intense pulsed light (syn. IPL), electrosurgery, and other relevant ingredients listed herein.

### Wound Healing

For formulations of the present invention for use as wound healing agents, in addition to Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicus, the formulation of the present invention may further comprise, for example, one or more of established wound healing therapies which include, but are not limited to hydrocolloids (e.g. Duoderm), silvadene, alginates (e.g. Sorbsan), hydrogels (e.g. Vigilon), foam dressing (e.g. Flexzan, Allevyn and Vigifoam), hydrofibers (e.g. carboxymethyl cellulose), non-adherent fabrics (e.g. gauze, telfa pad, xeroform, hydrophobic, hydrophilic), occlusive or moisture retentive dressings (e.g. nonbiologic, foam, film, biologic, antimicrobial, cadexomer iodine, silver dressing), corticosteroids (e.g. clobetasol, betamethasone, triamcinolone, hydrocortisone, prednisone), keratolytics (e.g. urea, salicylic acid, lactic acid, and other alpha- or beta-hydroxyacids), antiseptic agents including but not limited to alcohol, chlorhexidine (e.g. Hibiclens), iodine, iodophors (e.g. betadine), technicare PCMX chloroxylenol, triclosan, benzalkonium (quaternary ammonium), septisol, bromelain, antibiotics (e.g. clindamycin, metronidazole, mupirocin, gentamycin, erythromycin and retapamulin), anti-fungal agents (e.g. ketoconazole, nystatin, econazole, terbinafine, amphotericin, butenafine, naftifine and thymol), antivirals (e.g. acyclovir, valacyclovir, cidofovir, foscarnet, imiquimod, cantharidin and squaric acid), scabicides (e.g.

permethrin), pediculicides, anti-helminthics (e.g. benzoyl peroxide), silvadene, pentavalent antimony (e.g. stibogluconate), salicyclic acid, sulfur, sulfacetamide, anti-dandruff agents (e.g. pyrithione zinc, selenium sulfide, alkyl isoquinolinium bromide, allantoin, benzalkonium chloride), vinegar, collagen and derivates thereof (e.g. human, bovine, porcine, synthetic collagen), Autologen, dermalogen, fascian, isolagen, hyaluronic acid, silicone, liquid bandages (e.g. SkinStitch™), Pentoxifylline (syn. Trental), analgesics (e.g. lidocaine, prilocaine, pramoxine), acetic acid, aluminum acetate (e.g. Burow's solution), sodium hypochlorite (Dakin's solution), stanozolol and danazol, peptides (e.g. argireline and copper peptides), vitamins, antioxidants, emollients, calming agents, anti-pruritics, and other relevant ingredients listed herein. Additional herbal ingredients which can be included in formulations of the present invention for use as antimicrobial agents include, but are not limited to Hemidesmus indicus, Melaleuca alternifolia, Punica granatum, Momordica charantia, Terminalia chebula, Rosmarinus officinalis, Tephrosia purpurea, Terminalia arjuna, Yashada bhasma, Shorea robusta resin, Flax seed oil, Calendula, Crocus sativus, Symphytum officinale, Tridax procumbens, Trichosanthes, Bacopa monniera, Arctium lappa, Angelica dahurica, Picea abies (syn. Norway spruce), and Bacopa monnieri.

### Anti-Pruritics/Emollients/Xerosis

For formulations of the present invention for use as anti-pruritics, emmollients and/or in xerosis, in addition to Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicus, the formulation of the present invention may further comprise, for example, one or more of established therapies which include, but are not limited to Cocos nucifera (coconut oil), Sesamum indicum, Cucumis Satius, root extracts (e.g. ginger, gingko), dexpenthanol, silymarin, lipids, sterols, omega-3 fatty acids, ceramides, aloe vera, chamomile, calamine, camphor, menthol, eucalyptus, peppermint, capsaicin, antihistamines (e.g. diphenahydramine, hydroxyzine, cyproheptadine, loratidine, desloratadine, fexofenadine, cetirizine), H2-blockers (e.g. famotidine, cimetidine, ranitidine), pramoxine, doxepin, promethazine hydrochloride, antidepressants (e.g amitriptyline, TCA's, SSRI's, venlafaxine), antipsychotics (e.g. pimozide), naltrexone, ondansetron, cholestyramine, estrogen and estradiol and derivatives thereof, rifampin, coal tar, tar gel, tar shampoo, vitamins and minerals (e.g. calcipotriene, calcitriol, zinc, etc), opioids, anesthetics and analgesics (e.g. lidocaine, prilocaine, benzocaine, bupivicane, tetracaine, procaine, morphine, fentanyl), calcineurin inhibitors (e.g. tacrolimus, pimecrolimus), corticosteroids (e.g. clobetasol, betamethasone, triamcinolone, hydrocortisone, prednisone), keratolytics (e.g. urea, azelaic acid, salicylic acid, lactic acid, and other alpha- or beta- hydroxyacids), and other relevant ingredients listed herein.

### Disorders of Odor and Hyperhidrosis

For formulations of the present invention for use in disorders of odor and hyperhidrosis, in addition to Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicus, the formulation of the present invention may further comprise, for example, one or more of established therapies which include, but are not limited to, Cetylpyridinium chloride, menthol, thymol, methyl salicylate, eucalyptol, aluminum chloride (syn. Drysol), and glycopyrrolate (syn. Robinul), botulinum toxin, anticholinergics (e.g. atropine), calcineurin inhibitors (e.g. tacrolimus, pimecrolimus), corticosteroids (e.g. clobetasol, betamethasone, triamcinolone, hydrocortisone, prednisone), keratolytics (e.g. urea, azelaic acid, salicylic acid, lactic acid, and other alpha- or beta- hydroxyacids), retinol or retinoids and derivatives thereof (e.g., isotretinoin, acitretin, adapalene, tretinoin, tazorac), vitamin D analogs (e.g. calcitriol, calcipotriene), nicotinamide, gentian violet, liquor carbonis detergens, nitrogen mustard, bexarotene, alpha- or beta-agonists or antagonists (e.g. timolol or propranolol), oxymetazoline hydrochloride, aminolevulinic acid (syn. Levulan), emollients (e.g. ceramides), coal tar, tar gel, tar shampoo, antibiotics (e.g. clindamycin, metronidazole, mupirocin, polysporin, gentamycin), anti-fungal agents(e.g. ketoconazole, nystatin, econazole, terbinafine, griseofulvin, ciclopirox, miconazole, thymol, zeasorb, nafitine, amphotericin), antivirals (e.g. acyclovir, docosanol, penciclovir, valacyclovir, cidofovir, foscarnet, imiquimod, canthacur, squaric acid), scabicides (e.g. permethrin, ivermectin, lindane, malathion), pediculicides, anti-helmintic (e.g. Benzimidazoles, Diethylcarbamazine, Ivermectin, Pyrantel pamoate, Levamisole), resiquimod, benzoyl peroxide, silvadene, pentavalent antimony (e.g. stibogluconate), salicyclic acid, sulfur, sulfacetamide, pyrithione zinc, selenium sulfide, vinegar, anthralin, anti-tumor-necrosis-factor inhibitors (e.g. adalimumab, etanercept, infliximab), methotrexate, diaminodiphenyl sulfone (syn. dapsone), hydroxychloroquine (syn. plaquenil), chloroquine, quinacrine, azathioprine (syn. Imuran), anakinra (syn. Kineret), omalizumab (syn. Xolair), cyclosporine (syn. Neoral), cyclophosphamide (syn. Cytoxan), Mycophenolate mofetil (syn. Cellcept, Myfortic), thalidomide, rituximab, ustekinumab (syn. stelara), alefacept, leflunomide, Methoxypsoralen (syn. oxsoralen), immunoglobulins, sulfasalazine, mesalamine, brodalumab and other immunomodulators and immunosuppressants, and other relevant ingredients listed herein. Additional herbal ingredients which can be included in formulations of the present invention for use in disorders of odors and hyperhidrosis include, but are not limited to Andropogon jwarankus, Mesua ferrea, Terminalia chebula, Hemidesmus indicus, Melaleuca alternifolia, Hydrastis Canadensis, Mahonia aquifolium, Andrographis paniculata, Manuka honey (e.g. Methylglyoxal), Agastache rugosa, Dichroa febrifuga, Tridax procumbens, Lobelia chinensis, Ocimum basilicum, Melia azedarach, Aloysia triphylla, Acacia concinna, Rhus javanica, Picea abies (syn. Norway spruce), and Santalum album. In one embodiment, the composition is administered topically or intralesionally before, after or simultaneously with dermabrasion, ablative and non-ablative lasers (e.g. pulsed dye laser, Erb:YAG, CO2, Nd:YAG, Fraxel, Q-switched, Diode, Argon, Pulse excimer, Krypton, Copper), photodynamic therapy, intense pulsed light (syn. IPL), electrosurgery, chemical peeling agents (e.g. trichloroacetic acid, glycolic acid, salicyclic acid, recorcinol, lactic acid, phenol, croton oil) and other relevant ingredients listed herein.

### Disorders of Hypersensitivity and Autoimmunity

For formulations of the present invention for use in disorders of hypersensitivity and autoimmunity, in addition to Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicus, the formulation of the present invention may further comprise, for example, one or more of established therapies which include, but are not limited to, Pycnogenol, oatmeal, avenanthramides, bisabolol (syn. Levomenol), D- and L-Panthenol, azelaic acid, non-steroidal anti-inflammatory drugs (e.g. aspirin, ibuprofen, piroxicam), statins, chlorambucil, acetaminophen, allopurinol, hydroxyurea, penicillamine, cromolyn sodium, colchicine, diclofenac, retinol or retinoids and derivatives thereof (e.g., isotretinoin, acitretin, adapalene, tretinoin, tazorac), vitamin D analogs (e.g. calcitriol, calcipotriene), calcineurin inhibitors (e.g. tacrolimus, pimecrolimus), corticosteroids (e.g. clobetasol, betamethasone, triamcinolone, hydrocortisone, prednisone), keratolytics (e.g. urea, azelaic acid, salicylic acid, lactic acid, and other alpha- or beta- hydroxyacids), liquor carbonis detergens, nicotinamide, gentian violet, vincristine or vinblastine, nitrogen mustard, bexarotene, rapamycin, bleomycin, cidofovir, imatinib, oxymetazoline hydrochloride, hedgehog pathway inhibitors (e.g. vismodegib,syn. Erivedge™, emollients (e.g. ceramides), coal tar, tar gel, tar shampoo, rifampin, antibiotics (e.g., doxycycline, metronidazole, trimethoprim-sulfamethaxole, cephalexin), anthralin, anti-tumor-necrosis-factor inhibitors (e.g. adalimumab, etanercept, infliximab), methotrexate, diaminodiphenyl sulfone (syn. dapsone), hydroxychloroquine (syn. plaquenil), chloroquine, quinacrine, azathioprine (syn. Imuran), anakinra (syn. Kineret), omalizumab (syn. Xolair), cyclosporine (syn. Neoral), cyclophosphamide (syn. Cytoxan), Mycophenolate mofetil (syn. Cellcept, Myfortic), thalidomide, rituximab, ustekinumab (syn. stelara), alefacept, leflunomide, methoxypsoralen (syn. oxsoralen), immunoglobulins, sulfasalazine, mesalamine, brodalumab and other immunomodulators and immunosuppressants. Additional herbal ingredients which can be included in formulations of the present invention for use in disorders of hypersensitivity and autoimmunity include, but are not limited to, Fritillaria thunbergii, Rehmannia glutinosa, Terminalia chebula, Embelia ribes, Vitex negundo, Picrorhiza kurroa, Pterocarpus santalinus, Punica granatum, Terminalia arjuna, Tinospora cordifolia, Rubia cordifolia, Eclipta alba (syn. eclipta prostrata), Rosmarinus officinalis, Boswellia serrate, Citrus aurantium, Cinnamomum cassia, Trigonella foenum-graecum, Pyrus, Lonicera caerulea, Vaccinium myrtillus, Diosgenin, Cocos nucifera, Mahonia aquifolium, Andrographis paniculata, Aegle marmelos, Fritillaria thunbergii, Calendula, Crocus sativus, Coptis, Cardamom (genera Elettaria and Amomum), Dichroa febrifuga, Leonurus japonicus, Ligusticum wallichii (tetramethylpyrazine), Lobelia chinensis, Phellodendron amurense, Rehmannia glutinosa, Eucommia ulmoides, Gleditsia sinensis, Trichosanthes, Melia azedarach, Astragalus membranaceus, Platycodon graniflorus Arctium lappa, Forsythia suspense, Salvia miltiorrhiza Leonurus sibiricus, Mesua ferrea, Malabathrum, Bacopa monnieri, Broussonetia kazinoki, Perilla frutescens, Morus bombycis, Camellia sinensis, Glycyrrhiza glabra and Tanacetum parthenium. In one embodiment, the composition is administered topically or intralesionally before, after or simultaneously with ultraviolet B radiation, ultraviolet A radiation, photodynamic therapy, dermabrasion, ablative and non-ablative lasers (e.g. pulsed dye laser, Erb:YAG, CO2, Nd:YAG, Fraxel, Q-switched, Diode, Argon, Pulse excimer, Krypton, Copper, V-beam), intense pulsed light (syn. IPL), electrosurgery, and other relevant ingredients listed herein.

### Disorders of Angiogenesis

For formulations of the present invention for use in angiogenic disorders, in addition to Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicus, the formulation of the present invention may further comprise, for example, one or more of established angiogenic therapies which include, but are not limited to vasoconstrictors (e.g. catecholamines, oxymetazoline, phenylephrine, caffeine, theophylline, epinephrine, ergot compounds, triptans), penicillamine, tetracyclines, cytokines (e.g. IL-12), Matrix metalloproteinase inhibitors (e.g. batimastat and marimastat), direct angiogenesis inhibitors (e.g. bevacizumab, vascular endothelial growth factor receptor antagonists, angiostatin, basic fibroblast growth factor), indirect angiogenesis inhibitors (e.g. Ras-signaling inhibitors, farnesyltranferase inhibitors), sclerosing agents (e.g. hypertonic saline, sodium tetradecyl sulfate, SOTRADECOL™, polidocanol, sodium morrhuate, ethanolamine oleate, glycerin, polyiodine iodine), and other relevant ingredients listed herein. Additional herbal ingredients which can be included in formulations of the present invention for use in angiogenic disorders include, but are not limited to, Scutellaria barbata, Lobelia chinensis, Gleditsia sinensis and Melia zedarach.

Topical, intralesional and oral formulations for use in the present invention can be prepared by methods and contain carriers which are well-known in the art. A generally recognized compendium of such methods and ingredients is Remington: The Science and Practice of Pharmacy, Alfonso R. Gennaro, editor, 20th ed. Lippingcott Williams & Wilkins: Philadelphia, Pa., 2000.

Formulations suitable for application to a cutaneous or mucosal surface topically, intralesionally or orally can take the form of an ointment, cream, lotion, solution, paste (e.g.

facial mask), gel, emulsion, spray, aerosol, oil, patch, toothpaste, mouthwash, deodorant, soap, body and/or face wash, sponge, foam, semi-solid, cosmetic (e.g. solid, semisolid, liquid, or powder foundation, eye shadow, lip balm), application stick, sunscreen, depot, suppository, sustained-release formulation (a unique variation of this concept presented herein involves tea bag-like carriers made of plastic, silk, nylon, Soilon or paper), troche, tablet, pill, capsule, syrup, elixir, suspension, wafer, food (e.g. chewing gum, mints, etc.), beverage or other formulation which accomplishes direct contact between the composition of the present invention and a cutaneous or mucosal surface or lesion. Formulations can also be prepared which are suitable for collusive therapy.

Such formulations should contain at least 0.1% of Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicus. The percentage of the ingredient or ingredients and preparations can, of course, be varied and can conveniently be between about 0.1 to about 100% of the weight of a given unit dosage form. The amount of each ingredient in such compositions is such that an effective dosage level will be obtained.

Formulations in the forms of ointments, creams, lotions and pastes can generally have carriers in the forms of oleaginous bases (e.g., White Petrolatum and White Ointment); absorption bases formed by adding a water-in-oil emulsifying agent to an oleaginous base (e.g., Hydrophilic Petrolatum, AQUABASE, and AQUAPHOR); water-in-oil emulsion bases, prepared by adding water to an absorption base (e.g., HYDROCREAM, EUCERIN, NIVEA, and Cold Cream); oil-in-water emulsion bases (e.g., DERMABASE, UNIBASE, VELVACHOL, and hydrophilic ointment); water soluble bases (e.g., polyethylene glycol ointment such as PEG 400-600 G or PEG 3350-400 G); and any other commercially available compounding base. Suitable carriers to produce a spray, gel, solution, aerosol, emulsions, foam, semisolids, soap, wash, shampoo, deodorant, makeup foundation, lip balms, mouthwash, or toothpaste are well-known in the art.

A carrier for topical or intralesional application can also contain additional ingredients such as other carriers, moisturizers, humectants, emollients, dispersants, radiation blocking compounds (chemical or physical blockers), cleansing agents, wetting agents, emulsifiers, lubricants (e.g. sodium lauryl sulfate and magnesium stearate), as well as coloring agents, release agents, coating agents, sweetening, flavoring, and perfuming agents, preservatives, flavonoids, and antioxidants, anti-microbial agents (e.g. antibiotics, fungicides, scabicides, pediculicides, benzoyl peroxide, salicyclic acid, sulfur, sulfacetamide, pyrithione zinc), anti-inflammatory agents (e.g. corticosteroids, calcineurin inhibitors), keratolytics (agents that soften, loosen, and facilitate exfoliation of the squamous cells of the epidermis; e.g. urea or ammonium lactate), anti-photoaging (e.g. retinol or retinoids), anti-pigmentation agents (e.g. hydroquinone), anti-perspirants (e.g. aluminum chloride, aluminum zirconium), anti-neoplastic agents (e.g. imiquimod, 5-fluorauracil), anti-pruritics, or cooling and calming agents (e.g. calamine, camphor, menthol, capsaicin, antihistamines, coal tar), vitamins and minerals (e.g. calcipotriene, calcitriol, zinc, etc), root extracts (e.g. ginger, gingko), antioxidants (e.g. caffeine and caffeine salts, apigenin), hair-growth effectors (e.g. minoxidil, eflornithine, prostaglandin analogs or antagonists), flavoring agents, sweeteners, coloring agents, fragrances, dental products (e.g. whitening agents, fluoride), vasoconstrictors (e.g. caffeine, oxymetazoline hydrochloride, theophylline), agents that inhibit angiogenesis, pain relievers or anesthetics (e.g. lidocaine, benzocaine), as well as other suitable materials that do not have a significant adverse effect on the activity of the topical composition. Additional ingredients can include, for example a sodium acid phosphate moisturizer, witch hazel extract, glycerine humectant, apricot kernel oil emollient, corn oil dispersant, ceramides, or inositol.

For oral therapeutic administration, a composition of the present invention can be combined with one or more carriers and used in the form of, for example, ingestible tablets, buccal tablets, troches, capsules, elixirs, suspensions, syrups, wafers, chewing gums, mints, foods, beverages and the like.

In addition to Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicas, tablets, troches, pills, capsules, foods and energy drinks and the like can also contain the following: binders such as gum tragacanth, acacia, corn starch, or gelatin; excipients such as dicalcium phosphate; a disintegrating agent such as corn starch, potato starch, alginic acid and the like; a lubricant such as magnesium stearate; and a sweetening agent such as sucrose, fructose, lactose, sucralose, or aspartame or a flavoring agent such as peppermint, oil of wintergreen, or cherry flavoring. The above listing is merely representative and one skilled in the art could envision other binders, excipients, sweetening agents, flavoring agents, coloring agents, and fragrances, and the like. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac or sugar and the like.

Suspensions, syrups or elixirs can contain Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicus as well as sweetening agents (e.g. sucrose or fructose, preservative (e.g. methyl and propylparabens), dyes and flavoring agents (e.g. cherry or orange flavor), sterile diluents (e.g. water, saline, oils), buffers (e.g. acetates, citrates, or phosphates), chelating agents (e.g. ethylenediaminetetraacetic acid), agents for the adjustment of tonicity (e.g. dextrose, sodium chloride). Of course, any material used in preparing any unit dosage form should be substantially non-toxic in the amounts employed. In addition, Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicus can be incorporated into sustained-release preparations and devices including but not limited to, those relying on osmotic pressure diffusion gradients to obtain a desired release profile.

All the formulations presented in this patent for the Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicus may be prepared with carriers that protect the agent against rapid release, such as controlled release formulations, including implants, transdermal patches, microencapsulated (e.g. silicone matrices) or nanoparticle delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Alternatively, one can administer a composition containing Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicas in a local manner, for example, in a depot, suppository, or sustained-release formulation. A unique variation of this concept presented herein involves tea bag-like carriers made of plastic, silk, nylon, Soilon or paper.

Nonlimiting examples of materials which can serve as carriers in formulations of compositions of the present invention include sugars, such as lactose, glucose and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, coconut oil, and soybean oil; glycols, such as propylene glycol; polyols, such as glycerin, sorbitol, mannitol, and polyethylene glycol; esters, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in formulations.

In one embodiment, a topical formulation of the present invention further contains transdermal or skin penetrant enhancers. Suitable skin penetrant enhancers include, but are not limited to, solvents such as water, alcohols (e.g., methanol, ethanol, 2-propanol), alkyl methyl sulfoxides (e.g., dimethylsulfoxide, decylmethyl sulfoxide, tetradecyl methyl sulfoxide), pyrrolidones (e.g., 2-pyrrolidone, N-methyl-2-pyrrolidone, N-(2-hydroxyethyl)pyrrolidone), laurocapram (AZONE), and other solvents such as acetone, dimethyl acetamide, dimethyl formamide, tetrahydrofurfuryl alcohol; amphiphiles such as anionic surfactants (e.g., docusate sodium, sodium lauryl sulfate), cationic surfactants (e.g., quaternary ammonium salts), amphoteric surfactants (e.g., lecithins, cephalins, alkylbetamines), nonionic surfactants (mono-, di-, and triglycerides), and other fatty acids and alcohols (e.g., lauryl, cetyl, and stearyl alcohols), sucrose, sorbitan and PEG; urea and N,N-dimethyl-m-toluamide.

Formulations suitable for transdermal administration can be presented as discrete patches adapted to remain in intimate contact with the epidermis of the recipient for a prolonged period of time. There are different designs of the patch system that dictate release characteristics of the active agent and patch behavior: (i) matrix or monolithic and (ii) reservoir or membrane. In the matrix system, the inert polymer matrix binds with the active agent and controls its release from the device. In the reservoir system, the polymer matrix does not control release of the active agent. Instead, a rate-controlling membrane present between the drug matrix and the adhesive layer provides the rate-limiting barrier for release of the active agent from the device. It is contemplated that either patch system is suitable for delivery of the compositions disclosed herein. Formulations suitable for transdermal administration can also be delivered by iontophoresis (see, for example, Pharmaceutical Research 3 (6):318 (1986)) and typically take the form of an optionally buffered aqueous solution of the compound. Suitable formulations contain citrate or bis\tris buffer (pH 6) or ethanol/water.

Compositions of the present invention are administered topically, intralesionally, or orally to the subject in an effective amount.

By topical, intralesional and/or oral administration it is meant to be inclusive of application to cutaneous as well as mucosal surfaces.

By “effective amount” as defined herein, it is meant an amount of a composition of the presentation wherein the levels of Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicus are adjusted such that the composition, when topically or intralesionally applied, prevents, treats, alleviates symptoms and/or mitigates a selected skin disorder and/or disease. In embodiments of the present invention comprising one or more additional pharmaceutical ingredients established for use in a skin disorder or disease, the levels of Albizia lebbeck and/or Sophora flavescens and/or Hibiscus rosa sinensis and/or Phyllanthus emblica (syn. Emblica officinalis) and/or Azadirachta indica and/or Centella asiatica and/or Ganoderma and/or Rubia cordifolia and/or Scutellaria baicalensis and/or Astragalus membranaceus and/or Ocimum tenuiflorum (syn. Ocimum sanctum) and/or Acorus and/or Artemisia and/or Rheum and/or Schisandra and/or Ophiopogon japonicus may be further adjusted so that the composition inclusive of the one or more additional ingredients, when topically, intralesionally, or orally administered , prevents, treats, alleviates symptoms and/or mitigates a selected skin disorder and/or disease. Thus, by “effective amount” of a composition of the present invention, it is meant to include an amount sufficient to effect beneficial or desired results, including clinical results. As such, an effective amount of the compositions is one which includes, but is not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. Treatment can also mean prolonging survival as compared to expected survival if not receiving treatment. “Effective amount” is also meant to include levels which do not produce unwanted side effects including, but not limited to, redness, dryness, pain and/or pruritus.

Dosages for compositions of the present invention can be determined by methods known in the art, see, e.g., Remington: The Science and Practice of Pharmacy, Alfonso R. Gennaro, editor, 20th ed. Lippingcott Williams & Wilkins: Philadelphia, Pa., 2000 and can be administered for the prevention (i.e., before detectable signs or symptoms of a skin disorder or disease are observed) or treatment (i.e., after detectable signs or symptoms of a skin disorder are observed) of a skin disorder or disease. The selected effective dosage level will depend upon a variety of factors including the activity of the particular composition of the present invention employed, whether the composition is used for prevention or treatment of the skin disorder or disease, the formulation selected, the time of administration, the rate of excretion or metabolism of the particular composition being employed, the duration of the treatment, other drugs, compounds and/or materials used in combination with the particular composition employed, the age, sex, weight, condition, general health and prior medical history of the patient being treated, and like factors well-known in the medical arts.

A physician having ordinary skill in the art can readily determine and prescribe the effective amount of the composition of the present invention and the dosing regimen required for prevention or treatment of the skin disorder in a subject. By subject herein it meant to be inclusive of any mammalian subject for which prevention or treatment of a skin disorder or disease is sought.

The following nonlimiting examples further illustrate the instant claimed invention.

### EXAMPLES

### Example 1: Composition 1

A topical composition of the present invention, referred to herein as Composition 1, comprising as active ingredients Albizzia lebbeck (1%), schisandra chinensis (0.1%), and ophiopogon japonicus (2%), as well as petrolatum, water, glycerin, sodium hyaluronate, dimethicone, glyceryl stearate, acrylates, disodium EDTA, capric triglyceride, propanediol, phenoxyethanol, and triethanolamine, was prepared and administered to subjects as follows:

Subject 1 suffered from chronic eyelid eczema characterized by persistent severe itching and scaling of the eyelids. Subject 1 previously treated the affected area with emollients and hydrocortisone with minimal improvement. Subject 1 applied Composition 1 twice daily for 2 weeks with complete resolution of itching and scaling. Subject 1 sustained remission at 2 months follow up. Subject 1 did not experience any adverse effects during or after treatment with Composition 1.

Subject 2 suffered from arthropod hypersensitivity characterized by large, purpuric, intensely pruritic urticarial plaques which typically resolve with post-inflammatory hyperpigmentation and discoloration. Subject 2 applied Composition 1 twice daily (every 12 hours) for 2 days to these bug bites with complete resolution of itching, swelling, and redness within 3 days. The treated areas resolved without discoloration. The untreated areas were persistently pruritic, became purpuric, and ultimately resolved with post inflammatory hyperpigmentation and discoloration after 3 weeks. Subject 2 did not experience any adverse effects during or after treatment with Composition 1.

Subject 3 suffered from chronic scalp dermatitis characterized by persistent severe itching and scaling of the scalp. Subject 3 applied Composition 1 twice daily (every 12 hours) for 5 days to the scalp with significant reduction of itching and scaling of treated areas. Untreated areas were persistently itchy and scaly. In the past, Subject 3 treated these areas on the scalp with clobetasol 0.05% solution, an ultrapotent prescription topical steroid, the use of which was limited due to adverse side effects including acne and skin atrophy. Subject 3 achieved similar results with application of Composition 1 but without any adverse side effects.

Subject 4 suffered from stasis dermatitis characterized by recurrent pruritic eczematous dermatitis of the lower legs due to chronic lower leg swelling and varicose veins. Subject 4 applied Composition 1 to one leg and reported resolution of itching within 15 minutes of a single application and lasting for 5 days following treatment. Subject 4 reported persistent severe daily pruritus in the untreated leg. Subject 4 did not experience any adverse effects with Composition 1.

Subject 5 suffered from intermittent, episodic neuropathic pruritus. Prior to treatment with Composition 1, Subject 5 had on average of 4-5 episodes of intense pruritus per day with each episode lasting up to 2 hours. Subject 5 applied Composition 1 to affected areas and reported resolution of itching within 5-10 minutes of application. Subject 5 also reported fewer episodes, 1-2 episodes per day, after 2 days of using Composition 1. Subject 5 did not experience any adverse effects during treatment with Composition 1.

Subject 6 suffered from arthropod hypersensitivity characterized by purpuric and intensely pruritic urticarial plaques which typically resolve with post-inflammatory hyperpigmentation and discoloration. Subject 6 applied Composition 1 only twice to affected areas with resolution of itching. Subject 6 noted significant improvement of itching within 10 minutes after each application. Untreated bug bites persisted for 15 days with persistent itching, redness and ultimately resolved with post-inflammatory dark spots and discoloration. Subject did not experience any adverse effects with Composition 1.

Subject 7 suffered from pruritic eczematous patches and xerosis on the lower legs due to lack of emollient use and excessive washing with soap. Subject 7 applied Composition 1 to one leg twice daily for 3 days with improvement of dryness, scaling, and itching. Untreated areas on the legs and thigh continued to be persistently itchy and scaly despite emollient use. Subject 7 did not experience any adverse effects with composition 1.

### Example 2: Composition 2

A topical composition of the present invention, referred to herein as Composition 2, comprising as active ingredients Albizzia lebbeck (1%), phyllanthus emblica (0.5%), azadirachta indica (2%), and centella asiatica (0.1%), as well as petrolatum, water, glycerin, sodium hyaluronate, dimethicone, glyceryl stearate, acrylates, disodium EDTA, capric triglyceride, propanediol, phenoxyethanol, and triethanolamine, was prepared and administered to subjects as follows:

Subject 8 had a history of irritant contact dermatitis and sensitive skin. Subject 8 developed erythema, swelling, and tenderness after threading of her eyebrows and upper lip for hair removal. Subject 8 applied Composition 2 to the right eyebrow immediately after threading and the right half of the upper cutaneous lip immediately after hair removal. Subject 8 had significant improvement of erythema, swelling, and tenderness within 8 minutes of applying Composition 2 as compared to up to 1 hour for the untreated areas. Subject 8 did not experience any adverse effects with composition 2.

Subject 9 applied Composition 2 to acne papules on her face and had improvement ,of pain and redness within 12 hours of application. The treated papules did not evolve into pustules and completely resolved within 3-5 days without post-inflammatory hyperpigmentation (dark spots). Many of the untreated lesions on the face became pustular and resolved in 7-10 days with dark brown spots. The untreated lesions remained painful and red for up to 7 days. Subject 9 did not experience any adverse effects with application of Composition 2.

### Example 3: Composition 3

A topical composition of the present invention, referred to herein as Composition 3, comprising as active ingredients Albizzia lebbeck (1%), astragalus membranaceous (2%), ocimum sanctum (0.5%), and hibiscus rosa-sinensis (0.5%), as well as petrolatum, water, glycerin, sodium hyaluronate, dimethicone, glyceryl stearate, acrylates, disodium EDTA, capric triglyceride, propanediol, phenoxyethanol, and triethanolamine, was prepared and administered to subjects as follows:

Subject 10 developed superficial erosions on his lower legs after indoor rock climbing. Subject 10 applied Composition 3 twice daily for 3 contiguous days to a fresh wound on the left lower leg. The treated lesion re-epithelialized within 12 hours of application compared to 24-36 hours for untreated areas. At two weeks, the treated area resolved without post-inflammatory hyperpigmentation (darkening) while untreated areas developed gray-brown discoloration. Subject 10 did not experience burning or stinging or any other adverse effects with application of Composition 3. Neither site was infected during the study.

Subject 11 developed traumatic cuts on the left upper arm and left forearm from low lying shrubbery and branches. Subject 11 is skin type 4 and typically develops dark brown scars with cuts. Subject 11 applied Composition 3 immediately to the fresh wound on the left forearm, twice daily for 1 week. The treated area on the left forearm resolved in 1 week without forming a dark brown scar. The untreated area on her left upper arm resolved 3 weeks after the injury with a dark linear scar that was still present at 3 months follow up.

What is claimed is:

1. A composition comprising Albizia lebbeck and one or more agents selected from the group consisting of Sophora flavescens, Hibiscus rosa sinensis, Phyllanthus emblica (syn. Emblica officinalis), Azadirachta indica, Centella asiatica, Ganoderma, Rubia cordifolia, Scutellaria baicalensis, Astragalus membranaceus, Ocimum tenuiflorum (syn. Ocimum sanctum), Acorus, Artemisia, Rheum, Schisandra, and Ophiopogon japonicus.

2. A composition comprising Sophora flavescens and one or more agents selected from the group consisting of Albizia lebbeck, Hibiscus rosa sinensis, Phyllanthus emblica (syn. Emblica officinalis), Azadirachta indica, Centella asiatica, Ganoderma, Rubia cordifolia, Scutellaria baicalensis, Astragalus membranaceus, Ocimum tenuiflorum (syn. Ocimum sanctum), Acorus, Artemisia, Rheum, Schisandra, and Ophiopogon japonicus.

3. A composition comprising Hibiscus rosa sinensis and one or more agents selected from the group consisting of Albizia lebbeck, Sophora flavescens, Phyllanthus emblica (syn. Emblica officinalis), Azadirachta indica, Centella asiatica, Ganoderma, Rubia cordifolia, Scutellaria baicalensis, Astragalus membranaceus, Ocimum tenuiflorum (syn. Ocimum sanctum), Acorus, Artemisia, Rheum, Schisandra, and Ophiopogon japonicus.

4. A composition comprising Phyllanthus emblica (syn. Emblica officinalis) and one or more agents selected from the group consisting of Albizia lebbeck, Sophora flavescens, Hibiscus rosa sinensis, Azadirachta indica, Centella asiatica, Ganoderma, Rubia cordifolia, Scutellaria baicalensis, Astragalus membranaceus, Ocimum tenuiflorum (syn. Ocimum sanctum), Acorus, Artemisia, Rheum, Schisandra, and Ophiopogon japonicus.

5. A composition comprising Azadirachta indica and one or more agents selected from the group consisting of Albizia lebbeck, Sophora flavescens, Hibiscus rosa sinensis, Phyllanthus emblica (syn. Emblica officinalis), Centella asiatica, Ganoderma, Rubia cordifolia, Scutellaria baicalensis, Astragalus membranaceus, Ocimum tenuiflorum (syn. Ocimum sanctum), Acorus, Artemisia, Rheum, Schisandra, and Ophiopogon japonicus.

6. A composition comprising Centella asiatica and one or more agents selected from the group consisting of Albizia lebbeck, Sophora flavescens, Hibiscus rosa sinensis, Phyllanthus emblica (syn. Emblica officinalis), Azadirachta indica, Ganoderma, Rubia cordifolia, Scutellaria baicalensis, Astragalus membranaceus, Ocimum tenuiflorum (syn. Ocimum sanctum), Acorus, Artemisia, Rheum, Schisandra, and Ophiopogon japonicus.

7. A composition comprising Ganoderma and one or more agents selected from the group consisting of Albizia lebbeck, Sophora flavescens, Hibiscus rosa sinensis, Phyllanthus emblica (syn. Emblica officinalis), Azadirachta indica, Centella asiatica, Rubia cordifolia, Scutellaria baicalensis, Astragalus membranaceus, Ocimum tenuiflorum (syn. Ocimum sanctum), Acorus, Artemisia, Rheum, Schisandra, and Ophiopogon japonicus.

8. A composition comprising Rubia cordifolia and one or more agents selected from the group consisting of Albizia lebbeck, Sophora flavescens, Hibiscus rosa sinensis, Phyllanthus emblica (syn. Emblica officinalis), Azadirachta indica, Centella asiatica, Ganoderma, Scutellaria baicalensis, Astragalus membranaceus, Ocimum tenuiflorum (syn. Ocimum sanctum), Acorus, Artemisia, Rheum, Schisandra, and Ophiopogon japonicus.

9. A composition comprising Scutellaria baicalensis and one or more agents selected from the group consisting of Albizia lebbeck, Sophora flavescens, Hibiscus rosa sinensis, Phyllanthus emblica (syn. Emblica officinalis), Azadirachta indica, Centella asiatica, Ganoderma, Rubia cordifolia, Astragalus membranaceus, Ocimum tenuiflorum (syn. Ocimum sanctum), Acorus, Artemisia, Rheum, Schisandra, and Ophiopogon japonicus.

10. A composition comprising Astragalus membranaceus and one or more agents selected from the group consisting of Albizia lebbeck, Sophora flavescens, Hibiscus rosa sinensis, Phyllanthus emblica (syn. Emblica officinalis), Azadirachta indica, Centella asiatica, Ganoderma, Rubia cordifolia, Scutellaria baicalensis, Ocimum tenuiflorum (syn. Ocimum sanctum), Acorus, Artemisia, Rheum, Schisandra, and Ophiopogon japonicus.

11. A composition comprising Ocimum tenuiflorum (syn. Ocimum sanctum) and one or more agents selected from the group consisting of Albizia lebbeck, Sophora flavescens, Hibiscus rosa sinensis, Phyllanthus emblica (syn. Emblica officinalis), Azadirachta indica, Centella asiatica, Ganoderma, Rubia cordifolia, Scutellaria baicalensis, Astragalus membranaceus, Acorus, Artemisia, Rheum, Schisandra, and Ophiopogon japonicus.

12. A composition comprising Acorus and one or more agents selected from the group consisting of Albizia lebbeck, Sophora flavescens, Hibiscus rosa sinensis, Phyllanthus emblica (syn. Emblica officinalis), Azadirachta indica, Centella asiatica, Ganoderma, Rubia cordifolia, Scutellaria baicalensis, Astragalus membranaceus, Ocimum tenuiflorum (syn. Ocimum sanctum), Artemisia, Rheum, Schisandra, and Ophiopogon japonicus.

13. A composition comprising Artemisia and one or more agents selected from the group consisting of Albizia lebbeck, Sophora flavescens, Hibiscus rosa sinensis, Phyllanthus emblica (syn. Emblica officinalis), Azadirachta indica, Centella asiatica, Ganoderma, Rubia cordifolia, Scutellaria baicalensis, Astragalus membranaceus, Ocimum tenuiflorum (syn. Ocimum sanctum), Acorus, Rheum, Schisandra, and Ophiopogon japonicus.

14. A composition comprising Rheum and one or more agents selected from the group consisting of Albizia lebbeck, Sophora flavescens, Hibiscus rosa sinensis, Phyllanthus emblica (syn. Emblica officinalis), Azadirachta indica, Centella asiatica, Ganoderma, Rubia cordifolia, Scutellaria baicalensis, Astragalus membranaceus, Ocimum tenuiflorum (syn. Ocimum sanctum), Acorus, Artemisia, Schisandra, and Ophiopogon japonicus.

15. A composition comprising Schisandra and one or more agents selected from the group consisting of Albizia lebbeck, Sophora flavescens, Hibiscus rosa sinensis, Phyllanthus emblica (syn. Emblica officinalis), Azadirachta indica, Centella asiatica, Ganoderma, Rubia cordifolia, Scutellaria baicalensis, Astragalus membranaceus, Ocimum tenuiflorum (syn. Ocimum sanctum), Acorus, Artemisia, Rheum, and Ophiopogon japonicus.

16. A composition comprising Ophiopogon japonicus and one or more agents selected from the group consisting of Albizia lebbeck, Sophora flavescens, Hibiscus rosa sinensis, Phyllanthus emblica (syn. Emblica officinalis), Azadirachta indica, Centella asiatica, Ganoderma, Rubia cordifolia, Scutellaria baicalensis, Astragalus membranaceus, Ocimum tenuiflorum (syn. Ocimum sanctum), Acorus, Artemisia, Rheum, and Schisandra.

17. , A topical, intralesional, or oral formulation comprising any of the compositions of claims 1-16 and a topically, intralesionally or orally acceptable carrier.

18. A method for treating or alleviating symptoms of a skin disease or disorder, said method comprising topically, intralesionally, or orally administereing to a subject the formulation of claim 17.
